(19) United States
(12) Patent Application Publication
(10) Pub. No.: US 2023/0265136 A1
(43) Pub. Date: Aug. 24, 2023

(54) USE OF CNPY3 PROTEIN AS TARGET FOR TREATING OF DENGUE FEVER

(71) Applicant: Army Medical University, Chongqing (CN)

(72) Inventors: Jintao Li, Chongqing (CN); Xiaoyan Ding, Chongqing (CN); Yuxin Zhou, Chongqing (CN); Jiuxiang He, Chongqing (CN); Xiaoyang Zhou, Chongqing (CN); Minyue Qiu, Chongqing (CN)

(21) Appl. No.: 18/063,499

(22) Filed: Dec. 8, 2022

(30) Foreign Application Priority Data
Feb. 24, 2022 (CN) 202210176623.9

(51) Int. Cl. C07K 14/47; A61P 31/14

(52) U.S. Cl. CPC C07K 14/47; A61P 31/14; A61K 38/00

(57) ABSTRACT

A use of a canopy fibroblast growth factor signaling regulator 3 (CNPY3) protein as a target for treating of dengue fever is provided belongs to the technical field of antiviral drugs. An important host protein CNPY3 related to dengue virus infection is identified. A host gene is significantly down-regulated in blood of dengue patients and in dengue virus (DENV) infected dendritic cells and THP-1 cells. Its expression is negatively correlated with the progression of dengue disease and positively correlated with the expression of most Toll-like receptors. Down-regulation of the host gene inhibits the production of IFN-β and the expression of ISGs in THP-1 cells, promoting DENV-2 infection. Up-regulation of the host gene expression in Vero and HEK 293T cells inhibits DENV-2 infection. It indicates that CNPY3 participates in an innate immune response signaling pathway, has the effect of anti-dengue virus, and is a potential therapeutic target for dengue fever.

### TECHNICAL FIELD

The disclosure relates to the technical field of antiviral drugs, and more particularly to a use/application of a canopy fibroblast growth factor (FGF) signaling regulator 3 (CNPY3) protein as a target for treating of dengue fever.

### STATEMENT REGARDING SEQUENCE LISTING

The sequence listing associated with this application is provided in text format in lieu of a paper copy and is hereby incorporated by reference into the specification. The name of the XML file containing the sequence listing is 22100TBYX-USP1-MF-2022-0107-SL.xml. The XML file is 40,212 bytes; is created on Nov. 16, 2022; and is being submitted electronically via EFS-Web.

### BACKGROUND

Dengue fever is an acute infectious disease caused by dengue virus (DENV). About 50 to 100 million people are infected with the dengue virus each year. Dengue virus infection can cause clinical symptoms ranging from dengue fever (DF) to dengue hemorrhagic fever (DHF) or dengue shock syndrome (DSS). DHF or DSS is characterized by severe hemorrhage and damage to vital organs with high mortality, but its pathogenesis is still unclear. Due to the unique pathogenesis of the dengue virus infection, there are no vaccines and antiviral drugs that can be widely used to prevent or treat dengue fever. Dengvaxia® (also referred to as CYD-TDV), developed by Sanofi Pasteur Inc, is the first vaccine licensed for use in approximately 20 dengue endemic countries in Asia, Latin America, Oceania, and Europe. The vaccine, which is available to individuals 9 to 45 years of age living in the dengue endemic countries and areas, protects against serotypes 1 and 2 less than serotypes 3 and 4, and is not available to people who are most vulnerable to severe dengue-related symptoms. Therefore, due to the blocked development of dengue vaccines, the development of target drugs for dengue virus is particularly important.

From the perspective of host-pathogen interaction, the viremia of most DENV-infected individuals is controlled by innate and adaptive immune systems at an early stage. Innate antiviral immunity plays an important role in defending against viral pathogens and building adaptive immune responses. Pattern recognition receptor (PRR), a representative immune receptor in innate immunity, can recognize a pathogen-associated molecular patterns (PAMP). When the pattern recognition receptors detect pathogen-associated molecules, these detection signals can activate various transcription factors and promote the production of antiviral proteins such as type I and III interferons (IFNs). Toll-like receptors (TLRs), an important family of PRR, regulate innate immunity. TLRs can recognize and aggregate a variety of PAMPs into mitochondrial antiviral-signaling proteins (MAVS), which in turn stimulate the production of interferon regulatory factor 3 (IRF3), nuclear factor kappa light chain enhancer of activated B cells (NF-κB), and type I IFN. Many studies have shown that the dengue virus can hide and mask its external molecular features after evolution, regulate TLR signaling pathways at multiple levels, inhibit antiviral responses, and thus promote viral replication and spreading. It is found that a canopy fibroblast growth factor signaling regulator 3 (CNPY3) protein is related to the pathogenesis and immune escape of the dengue virus, and the CNPY3 protein combined with members of TLRs as a chaperone to help the member proteins fold and export. In addition, it is found that CNPY3 has an anti-dengue virus function by in vitro infection of mouse models, and is a key regulator factor of the host innate immune system during DENV infection and a potential therapeutic target.

### SUMMARY

A technical problem to be solved by the disclosure is to provide a new option for treating dengue virus infection.

Technical solutions of the disclosure are as follows. In an aspect, the disclosure provides a use of a canopy fibroblast growth factor signaling regulator 3 (CNPY3) protein as a target for treating of dengue fever.

In another aspect, the disclosure also provides a use of upregulating an expression of a CNPY3 protein in an anti-dengue virus.

Specifically, an amino acid sequence of the CNPY3 protein is shown in SEQ ID NO: 1.

In an embodiment, a nucleotide sequence of an encoding gene of the CNPY3 protein is as shown in SEQ ID NO: 2 or SEQ ID NO: 45.

In still another aspect, the disclosure also provides an anti-dengue virus drug, mainly includes at least one of a messenger ribonucleic acid (mRNA) of CNPY3, a CNPY3 protein, a carrier expressing CNPY3 protein, and a host cell expressing CNPY3 protein.

Specifically, an amino acid sequence of the CNPY3 protein is shown in SEQ ID NO: 1.

In an embodiment, a nucleotide sequence of an encoding gene of the CNPY3 protein is shown in SEQ ID NO: 2 or SEQ ID NO: 45.

In an embodiment, the mRNA of the CNPY3 protein is encapsulated with lipid nanoparticles (LNPs).

Specifically, the mRNA of the CNPY3 protein is cloned into a T/A carrier, transcribed in vitro after linearization, added with a cap structure (m7GpppN) at a 5′-terminal and an A-tailing at a 3′-terminal of the transcribed mRNA, and encapsulated with the LNPs.

SEQ ID NO: 1 represents the amino acid sequence of the CNPY3 protein, and is specifically shown as follows:

MDSMPEPASRCLLLLPLLLLLLLLLPAPELGPSQAGAEENDWVRLPSKCE
VCKYVAVELKSAFEETGKTKEVIGTGYGILDQKASGVKYTKSDLRLIEVT
ETICKRLLDYSLHKERTGSNRFAKGMSETFETLHNLVHKGVKVVMDIPYE
LWNETSAEVADLKKQCDVLVEEFEEVIEDWYRNHQEEDLTEFLCANHVLK
GKDTSCLAEQWSGKKGDTAALGGKKSKKKSSRAKAAGGRSSSSKQRKELG
GLEGDPSPEEDEGIQKASPLTHSPPDEL.

In the amino acid sequence of the CNPY3 protein, M represents methionine abbreviated Met, D represents aspartic acid abbreviated Asp, S represents serine abbreviated Ser, P represents proline abbreviated Pro, E represents glutamic acid abbreviated Glu, A represents alanine abbreviated Ala, R represents arginine abbreviated Arg, C represents cysteine abbreviated Cys, L represents leucine abbreviated Leu, G represents glycine abbreviated Gly, Q represents glutamine abbreviated Gln, N represents asparagine abbreviated Asp, W represents tryptophane abbreviated Trp, V represents valine abbreviated Val, K represents lysine abbreviated Lys, Y represents tyrosine abbreviated Tyr, F represents phenylalanine abbreviated Phe, I represents isoleucine abbreviated Ile, T represents threonine abbreviated Thr, and H represents histidine abbreviated His.

In an embodiment, the nucleotide sequence of the encoding gene of the CNPY3 protein is shown in SEQ ID NO: 2.

SEQ ID NO: 2 represents a nucleotide sequence of an encoding gene of the CNPY3 protein, and is specifically shown as follows:

atggattcaatgcctgagcccgcgtcccgctgtcttctgcttcttccctt
gctgctgctgctgctgctgctgctgccggccccggagctgggcccgagcc
aggccggagctgaggagaacgactgggttcgcctgcccagcaaatgcgaa
gtgtgtaaatatgttgctgtggagctgaagtcagcctttgaggaaaccgg
caagaccaaggaggtgattggcacgggctatggcatcctggaccagaagg
cctctggagtcaaatacaccaagtcggacttgcggttaatcgaagtcact
gagaccatttgcaagaggctcctggattatagcctgcacaaggagaggac
cggcagcaatcgatttgccaagggcatgtcagagacctttgagacattac
acaacctggtacacaaaggggtcaaggtggtgatggacatcccctatgag
ctgtggaacgagacttctgcagaggtggctgacctcaagaagcagtgtga
tgtgctggtggaagagtttgaggaggtgatcgaggactggtacaggaacc
accaggaggaagacctgactgaattcctctgcgccaaccacgtgctgaag
ggaaaagacaccagttgcctggcagagcagtggtccggcaagaagggaga
cacagctgccctgggagggaagaagtccaagaagaagagcagcagggcca
aggcagcaggcggcaggagtagcagcagcaaacaaaggaaggagctgggt
ggccttgagggagaccccagccccgaggaggatgagggcatccagaaggc
atcccctctcacacacagcccccctgatgagctctga

The beneficial effects of the disclosure as follows. In order to identify genes related to DENV pathogenesis and find some new therapeutic targets, the disclosure uses the disclosed datasets to analyze genes related to dengue virus infection and identify an important host protein CNPY3. The host gene is significantly down-regulated in the blood and dengue virus infected dendritic cells and human leukemia monocytic cells (THP-1) of dengue patients. The expression of the CNPY3 is negatively correlated with the progression of dengue disease and positively correlated with the expression of most Toll-like receptors. Further studies show that down-regulation of the host gene in THP-1 cells can inhibit the production of interferon-beta (IFN-β) and the expression of interferon-stimulated genes (ISGs), and thus promote DENV-2 infection. However, up-regulation of the host gene expression in Vero cells and Human embryonic kidney 293T cells (HEK 293T, also known as a daughter cell line derived from the HEK293 original cell line) can inhibit the DENV-2 infection. These results indicate that CNPY3 participates in the innate immune response signaling pathway, has the effect of anti-dengue virus, and is a potential therapeutic target for dengue fever.

### BRIEF DESCRIPTION OF DRAWINGS

FIG. 1A illustrates a Venn diagram showing an intersection of common differentially expressed genes between a dataset of dengue virus (DENV)-infected human monocyte-derived dendritic cells and a transcriptome dataset of dengue patients.

FIG. 1B illustrates a heatmap results of functional enrichment analysis of the differentially expressed genes, showing top gene ontology (GO) biological processes, where discrete color marks indicate statistical significance.

FIG. 1C illustrates a heatmap showing consistently expressed genes in the dataset of DENV-infected human monocyte-derived dendritic cells and the transcriptome dataset of dengue patients.

FIG. 1D illustrates an expression verification of differentially expressed genes in human leukemia monocytic cells (THP-1) after DENV-2 infection through transcriptome analysis, where results are expressed as standard deviations (n=3), asterisks indicate significant differences (t-test, *, P<0.05; **, P<0.01; ***, p<0.001); MOCK represents blank; and DENV-1, DENV-2, DENV-3, and DENV-4 represent dengue virus types 1, 2, 3, and 4, respectively.

FIG. 2 illustrates an expression of a canopy fibroblast growth factor signaling regulator 3 (CNPY3) in DENV-2 infected suckling mice. 3-day-old Balb/C suckling mice are infected with 3 microliters (µL) of LDENV-2 (8×10⁶ plaque forming units per milliliter abbreviated PFU/mL) intranasally, and Mock mice are infected with 3 µL of phosphate buffered saline (PBS) intranasally. On the 7^{th} day after infection, ribonucleic acid (RNA) is extracted from brain and whole blood of the mice. In the figure, Mock represents a control group not infected with DENV-2; Infected represents tissues infected with DENV-2 for 7 days, results are expressed as standard deviations (n=3), and asterisks indicate significant differences (t-test, *, P<0.05; **, P<0.01; ***, P<0.001).

FIG. 3A illustrates an expression of CNPY3 of whole blood in the DENV-2 infected suckling mice on the 2^{nd} day and the 4^{th} day after infection, where Infected represents 3-day-old Balb/C suckling mice injected intracranially with 20 µL (60 PFU) of DENV-2; Mock represents 3-day-old Balb/C suckling mice injected intracranially with 20 µL of PBS, and results are expressed as standard deviations (n=3).

FIG. 3B illustrates an expression of CNPY3 in DENV-infected human monocyte-derived dendritic cells at different time points, where raw data is derived from a public dataset GSE58278, and Uninfected represents a control group.

FIGS. 3C-3D illustrate an expression of CNPY3 in dengue patients with different disease progression, where raw data is derived from public datasets GSE18090 and GSE51808, asterisks represent significant differences (t-test, *, P<0.05; **, P<0.01; ***, P<0.001), DF represents dengue fever, and DHF represents dengue hemorrhagic fever.

FIG. 4A illustrates expressions of RNA and protein of CNPY3 in human tissues analyzed by using a Human Platelet Antigen (HPA) database.

FIG. 4B illustrates expressions of CNPY3 in blood cells analyzed by using the Human Platelet Antigen (HPA) database.

FIG. 4C illustrates expressions of CNPY3 at a single-cell level in blood, where single-cell data includes scRNA-seq data from different peripheral blood mononuclear cells (PBMCs) and endothelial cell clusters.

FIGS. 5A-5J illustrate a correlation analysis of the expression of CNPY3 and expressions of Toll-like receptors (TLRs) including TLR1, TLR2, TLR3, TLR4, TLR5, TLR6, TLR7, TLR8, TLR9 and TLR10 in healthy human blood.

FIGS. 6A-6J illustrate an expression analysis of the Toll-like receptors in dengue patients, where Control represents a control group.

FIGS. 7A-7B illustrate expressions mRNA and protein of CNPY3. THP-1 cells are transfected with small interfering RNA (siRNA) targeting CNPY3 or negative control (NC) siRNA, and the THP-1 cells are collected after 36 hours to determine the relative expressions of CNPY3. β-actin is used as an internal reference gene, NC represents THP-1 cells transfected with negative control siRNA, siRNA represents THP-1 cells transfected with siRNA targeting CNPY3, the expression of NC-siRNA targeting CNPY3 is used as a control group, and results are expressed as standard deviations (n=3), Student’s t test, *, P<0.05; ***, P<0.001.

FIG. 7C illustrates a content of interferon-beta (IFN-β) detected by enzyme-linked immunosorbent assay (ELISA).

FIG. 7D illustrates an expression analysis of interferon-stimulated genes (ISGs).

FIG. 8A illustrates relative expressions of envelope proteins (also referred to as E proteins) and non-structural (NS) proteins NS3 and NS5 of DENV-2 detected after continuous culture for 18 hours. β-actin is used as an internal reference gene, NC represents THP-1 cells transfected with negative control siRNA, siRNA represents THP-1 cells transfected with siRNA targeting CNPY3, and results are expressed as standard deviations (n=3), t-test, *, P<0.05.

FIG. 8B illustrates a copy number (also referred to as copies) of virus in a cell supernatant detected after continuous culture for 2d, 4d and 6d.

FIG. 8C illustrates a content of the E proteins in the cell supernatant after culture for 6 days.

FIG. 9A illustrates a number of DENV-2 infected Vero cells after transfection of pcDNA3.1 (i.e., empty vector control) or pcDNA3.1-CNPY3 observed by a fluorescence microscope. The plasmids pcDNA3.1 and pcDNA3.1-CNPY3 are transfected into Vero cells at a same concentration for 36 hours, and then the Vero cells are incubated with DENV-2 (multiplicity of infection abbreviated MOI=2) for 2 hours, a virus solution is discarded, and cultured continuously for 18 hours. Specimens are fixed, permeated, stained, and imaged. DAPI represents 4′,6-diamidino-2-phenylindole, anti-dengue represents anti-dengue antibodies, and Uninfected represents uninfected cells.

FIG. 9B illustrates a quantitative analysis of the number of DENV-2 infected Vero cells. The DENV-2 infected Vero cells for each experimental condition are counted using an Image J software in eight different regions. Results are expressed as standard deviations (n=3), t-test, *, P<0.05; **, P<0.01; ***, P<0.001.

FIG. 9C illustrates relative expressions of the E proteins and NS3 and NS5 in the DENV-2 infected Vero cells, wherein β-actin is used as the reference gene.

FIGS. 9D-9E illustrate expressions of mRNA and protein of CNPY3 detected by real-time quantitative polymerase chain reaction (RT-qPCR) and western blot. HEK 293T cells are transfected with pcDNA3.1 (i.e., empty vector control) or pcDNA3.1-CNPY3 for 36 hours, the expressions of CNPY3 are detected by the RT-qPCR and western blot.

FIG. 9F illustrates relative expressions of the E proteins and NS3 and NS5 in the HEK 293T cells infected with DENV-2, where β-actin is used as the reference gene.

FIG. 9G illustrates copies in a supernatant of the HEK 293T cells infected with DENV-2 for 6 days after overexpression of CNPY3.

FIG. 9H illustrates an infection of Vero cells observed by immunofluorescence. HEK 293T cells are transfected with pcDNA3.1 (i.e., empty vector control) or pcDNA3.1-CNPY3 for 36 hours, incubated with DENV-2 (MOI=2) for 2 hours, the virus solution is discarded, and cultured continuously for 6 days. Then, virus in a cell supernatant is collected and used to infect Vero cells, and immunofluorescence is performed 18 hour later.

FIG. 10 illustrates results of survival statistics. 10 µL of DENV-2 (60PFU) is tanken and mixed with 10 µL of empty vector lipid nanoparticles (LNPs) and a CNPY3 preparation, and then injected intracranially into 3-day-old suckling mice simultaneously, and a mortality rate is counted daily.

### DETAILED DESCRIPTION OF EMBODIMENTS

The disclosure is described below in combination with the drawings.

### Embodiment 1 Screening of Host Differentially Expressed Genes During Dengue Virus (DENV) Infection

A DENV-infected dendritic cells chip dataset GSE58278 of the Gene Expression Omnibus (GEO) (https: //www.ncbi.nlm.nih.gov/geo/query/acc.cgi?Acc=GSE58278) is download. Using an online differential expression analysis tool GEO2R of the National Center for Biotechnology Information (NCBI), differentially expressed genes between DENV-2 infected cells after infection for 24 hours and uninfected cells in the dataset are analyzed and screened out the differentially expressed genes with p≤0.05 and Log2FC≥1, of which 387 genes are up-regulated and 295 genes are down-regulated.

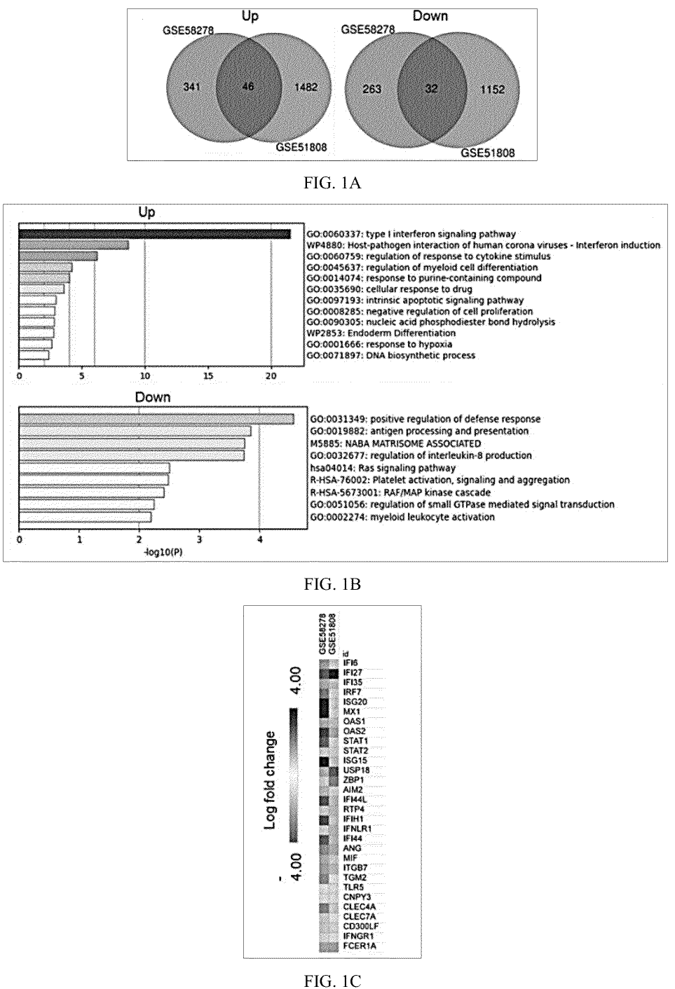

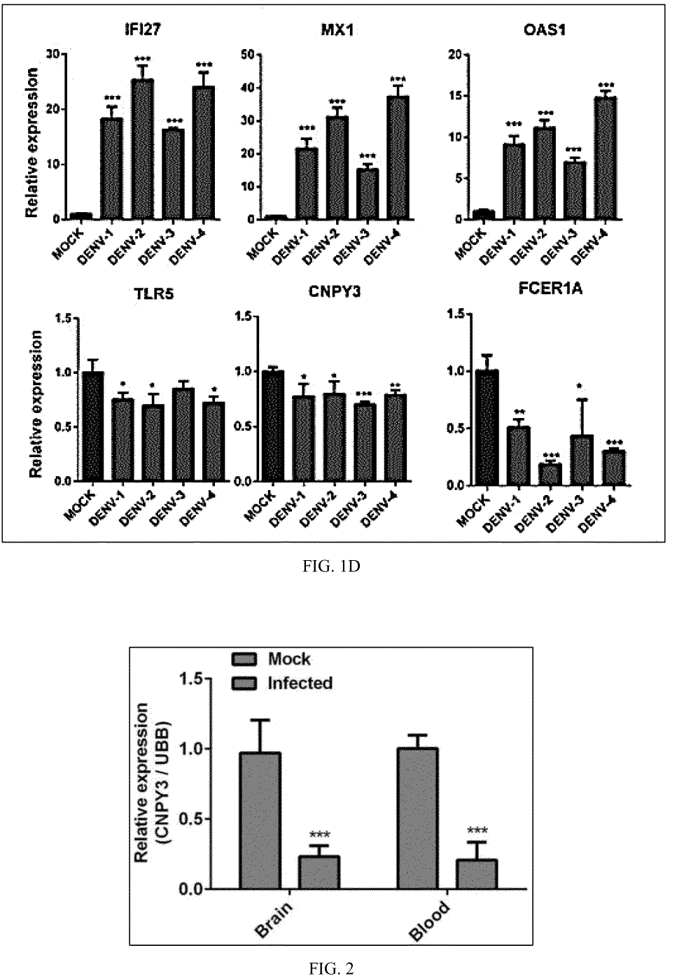

Combined with a transcriptome dataset GSE51808 of dengue patient (https: //www.NCBI.nlm.nih.gov/geo/query/acc.cgi), whole blood samples of 9 healthy people are used as a control group and whole blood samples of 10 dengue hemorrhagic fever patients are used as an infection group, the online differential expression analysis tool GEO2R of NCBI is used to screen out differentially expressed genes with p≤0.05 and Log2FC≥1. The sample numbers of healthy people are GSM1253075 to GSM1253083, and those of hemorrhagic fever patients are GSM1253032, GSM1253034, GSM1253037, GSM1253039, GSM1253040 GSM1253041, GSM1253046, GSM1253048, GSM1253049, and GSM1253052. Finally, 1528 up-regulated genes and 1184 down-regulated genes are obtained. Venn analysis of common differentially expressed genes in the chip dataset of DENV-infected dendritic cells and the transcriptome dataset of dengue patients shows that a total of 46 common up-regulated genes and 32 common down-regulated genes (FIG. 1A). The biological function enrichment analysis results of the common differentially expressed genes (DEGs) show that the up-regulated genes such as USP18, MX1, OSA1, OSA2, ISG20, etc. are mainly concentrated in the interferon type I signaling pathway. Many reports have proved that the interferon type I signaling pathway is the main innate immunity signaling pathway for the body to clear pathogens. Up-regulation of genes related to type 1 interferon signaling pathway after dengue virus infection contributes to dengue virus clearance (FIGS. 1B and C). It is noteworthy that the genes FCER1A, IFNGR1, CD300LF, CLEC7A, CLEC4A, CNPY3, TLR5, TGM2, which are down-regulated at both cellular and human levels, participate in the biological functional pathway of positive regulation of defense response. Therefore, the downregulation of these genes may reduce the body’s defense against dengue virus infection (FIGS. 1B and C).

The expression levels of three up-regulated genes (IFI27, MX1 and OAS1) involved in interferon signaling pathway and three down-regulated genes (TLR5, CNPY3 and FCER1A) involved in positive regulation of defense response in DENV-infected human leukemia monocytic cell line (THP-1) are further verified by real-time quantitative polymerase chain reaction (RT-qPCR). After THP-1 cells are infected with DENV-1 DENV-2, DENV-3, and DENV-4 for 24 hours, the relative expression of these genes is detected. The results show that the genes IFI27, MX1 and OAS1 are significantly up-regulated and the genes TLR5, CNPY3 and FCER1A are significantly down-regulated after DENV infection compared with the control group without DENV infection (FIG. 1D). These results are consistent with the dataset analysis results and thus verify the differentially expressed genes during DENV infection.

### Embodiment 2 Canopy Fibroblast Growth Factor Signaling Regulator 3 (CNPY3) Expression Negatively Correlated With DENV Disease Severity

Among the genes analyzed in the embodiment 1, few studies have been conducted on the role of CNPY3 in DENV infection. Therefore, a mouse model of DENV-2 infection in suckling mice is used for further study.

The virus strains of DENV-1 (strain name ThD1-0102-01), DENV-2 (strain name New Guinea), DENV-3 (strain name 80-2) and DENV-4 (strain name GD07-78) used in the disclosure are provided by the Disease Control and Prevention Center of Guangzhou Military Region, China. DENV-1, DENV-2, DENV-3, and DENV-4 proliferate in Vero cells.

Experiment of dengue virus infection in suckling mice is as follows. 3-day-old BALB/c suckling mice (female BALB/c pregnant mice are purchased from the Experimental Animal Center of the Army Medical University, China) are intracranially injected with New Guinea DENV-2, DENV-2 virus are isolated from the brain of suckling mice (virus titer 8×10⁶ plaque forming units per milliliter abbreviated PFU/mL), 3 microliters (µL) of the isolated DENV-2 virus are taken to 3-day-old suckling mice for nasal drip, and ribonucleic acid (RNA) is extracted from the brain and blood of mice 7 days after infection. In the control group, 3 µL of the supernatant of the brain tissue of healthy mice is taken and dripped into the nose of 3-day-old suckling mice, and RNA is extracted from the same site 7 days later. RNA from collected cells and tissues of the suckling mice is extracted using a tissue/cell rapid extraction kit (BOER, China) in strict accordance with the instructions. Reverse transcription is subsequently performed using Prime Script™ RT Reagent Kit (TaKaRa, Japan). Fluorescent quantitative PCR detection is performed using TBGreen® Premix Ex Taq™II (TaKaRa, Japan) and LightCycler® 96System (Roche, USA). The amplification reaction system is shown in Table 1.

TABLE 1

Fluorescent quantitative PCR amplification reaction system

| | |
|---|---|
| Name | Volume |
| TB Green Premix Ex Taq II (Tli RNase Plus) (2×) | 5 µL |
| Forward primer (10 micromoles per liter abbreviated µM) | 0.3 µL |
| Reverse primer (10 µM) | 0.3 µL |
| Template | 1 µL |
| Double-distilled water (ddH₂O) without nuclease | Up to 10 µL |

Reaction conditions are as follows. Step 1, pre-denaturation 95 Celsius degree (°C) 30 seconds (s); denaturation at 95° C. for 5 s; step 2, 0 cycles; 60° C. for 30 s, and step 3, a reaction condition of dissociation curve: gradually rise to 95° C. After the completion of PCR reaction, whether the dissociation curve and amplification curve are abnormal is checked, and the experimental data is exported. The expression level of the target gene is calculated by -ΔΔct method. Primers used for target gene detection are shown in Table 2.

Related primers used in the embodiment of Table 2

| | | | |
|---|---|---|---|
| Name | Sequence Number | Primer Sequence (5′-3′) | Use |
| β-actin | SEQ IDNO: 3 | F: caccattggcaatgagcggttc | THP-1 cells, HEK293T cells, RT-qPCR |
| | SEQ IDNO: 4 | R: aggtctttgcggatgtccacgt | THP-1 cells, HEK293T cells, RT-qPCR |
| β-actin | SEQ IDNO: 5 | F: caccattggcaatgagcggttc | Vero cells, RT-qPCR |
| | SEQ IDNO: 6 | R: aggtctttacggatgtccacgt | Vero cells, RT-qPCR |
| IFI27 | SEQ IDNO: 7 | F: tgctctcacctcatcagcagt | THP-1 cells, RT-qPCR |
| | SEQ IDNO: 8 | R: cacaactcctccaatcacaact | THP-1 cells, RT-qPCR |
| MX1 | SEQ IDNO: 9 | F: gtttccgaagtggacatcgca | THP-1 cells, RT-qPCR |
| | SEQ IDNO: 10 | R: ctgcacaggttgttctcagc | THP-1 cells, RT-qPCR |
| OAS1 | SEQ IDNO: 11 | F: tgtccaaggtggtaaagggtg | THP-1 cells, RT-qPCR |
| | SEQ IDNO: 12 | R: ccggcgatttaactgatcctg | THP-1 cells, RT-qPCR |
| TLR5 | SEQ IDNO: 13 | F: ccttacagcgaacctcatccac | THP-1 cells, RT-qPCR |
| | SEQ IDNO: 14 | R: tccactacaggaggagaagcga | THP-1 cells, RT-qPCR |
| CNPY3 | SEQ IDNO: 15 | F: gtcaaggtggtgatggacatcc | THP-1 cells, RT-qPCR |
| | SEQ IDNO: 16 | R: ctgtaccagtcctcgatcacct | THP-1 cells, RT-qPCR |
| FCER1A | SEQ IDNO: 17 | F: gtggagaatacaaatgtcagcacc | THP-1 cells, RT-qPCR |
| | SEQ IDNO: 18 | R: ctccatcaccacctcagcagag | THP-1 cells, RT-qPCR |
| OAS2 | SEQ IDNO: 19 | F: gcttccgacaatcaacagccaag | THP-1 cells, RT-qPCR |
| | SEQ IDNO: 20 | R: cttgacgattttgtgccgctcg | THP-1 cells, RT-qPCR |
| ISG15 | SEQ IDNO: 21 | F: ctctgagcatcctggtgaggaa | THP-1 cells, RT-qPCR |
| | SEQ IDNO: 22 | R: aaggtcagccagaacaggtcgt | THP-1 cells, RT-qPCR |
| IRF7 | SEQ IDNO: 23 | F: ccacgctataccatctacctgg | THP-1 cells, RT-qPCR |
| | SEQ IDNO: 24 | R: gctgctatccagggaagacaca | THP-1 cells, RT-qPCR |
| USP18 | SEQ IDNO: 25 | F: tggacagacctgctgccttaac | THP-1 cells, RT-qPCR |
| | SEQ IDNO: 26 | R: ctgtcctgcatcttctccagca | THP-1 cells, RT-qPCR |
| UBB | SEQ IDNO: 27 | F: ttcggtctgcattcccagtg | Mouse, RT-qPCR |
| | SEQ IDNO: 28 | R: aacttaaattggggcaagtggc | Mouse, RT-qPCR |
| CNPY3 | SEQ IDNO: 29 | F: tggagctgaagtcggctttt | Mouse, RT-qPCR |
| | SEQ IDNO: 30 | R: cttgtgcaggctgtagtcca | Mouse, RT-qPCR |
| E protein | SEQ IDNO: 31 | F: aggagtagagccgggacaat | DENV-2, RT-qPCR |
| | SEQ IDNO: 32 | R: cgctcccctcattgttgtct | DENV-2, RT-qPCR |
| NS3 | SEQ IDNO: 33 | F: agcccatttcacagacccag | DENV-2, RT-qPCR |
| | SEQ IDNO: 34 | R: tgtccagaac tccacgaacg | DENV-2, RT-qPCR |
| NS5 | SEQ IDNO: 35 | F: gatgtagacctcggaagcgg | DENV-2, RT-qPCR |
| | SEQ IDNO: 36 | R: gtcagcagtctgaccactcc | DENV-2, RT-qPCR |
| NS1 | SEQ IDNO: 37 | F: gcagaatgccccaacacaaa | DENV-2, RT-qPCR |
| | SEQ IDNO: 38 | R: acaaaatacatcctgcctttctct | DENV-2, RT-qPCR |
| DV2 | SEQ IDNO: 39 | F: gcagaatgccccaacacaaa | DV2, RT-qPCR |
| | SEQ IDNO: 40 | R: acaaaatacatcctgcctttctct | DV2, RT-qPCR |

RT-qPCR results show that the expression of CNPY3 in brain and blood of the suckling mice is inhibited 7 days after DENV-2 infection (FIG. 2).

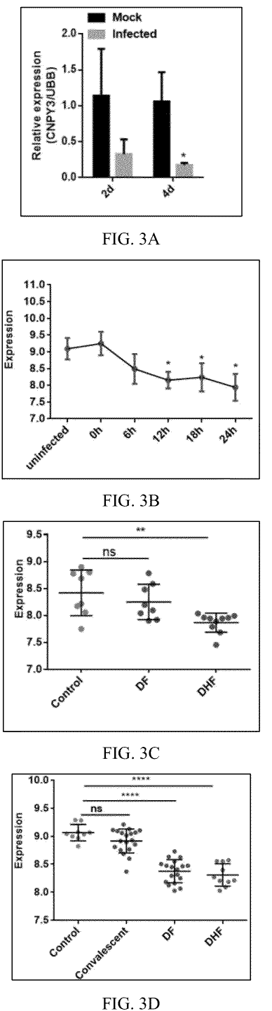

The expression of CNPY3 in the blood of the suckling mice is further detected, showing that with the prolongation of DENV-2 infection, the expression of CNPY3 in the blood is inhibited more significantly (FIG. 3A). Similarly, in the dataset GSE58278 (https://www.ncbi.nlm.nih.gov/geo/query/acc.cgi?Acc=GSE58278), the expression of CNPY3 decreases with the prolongation of infection time, especially at 12, 18 and 24 hours (FIG. 3B). Therefore, it is speculated that CNPY3 is related to the disease severity after DENV infection. DENV mediated diseases range in severity from asymptomatic infections and mild dengue fever (DF) to dengue hemorrhagic fever (DHF) and dengue shock syndrome (DSS). Using the publicly available transcriptome dataset GSE18090 (https://www.ncbi.nlm.nih.gov/geo/query/acc.cgi?Acc=GSE18090), the expression of CNPY3 is evaluated in peripheral blood mononuclear cells of a control group (fever patients confirmed not to be infected with dengue fever), mild dengue fever (DF) and dengue hemorrhagic fever (DHF) patients. The data shows that CNPY3 expression is significantly down-regulated in peripheral blood mononuclear cells of patients with severe dengue. The whole blood CNPY3 expressions of 28 dengue patients (DF n=18, DHF n=10), 19 convalescent patients (DF n=13, DHF n=6) and 9 healthy and non-infectious blood donors are further analyzed using the dataset GSE51808. The data shows that CNPY3 expression is negatively correlated with the severity of dengue fever (FIGS. 3C and 3D). These data indicates that CNPY3 plays an important role in the progression of DENV disease.

### Embodiment 3 CNPY3 Plays an Important Role in Toll-like Receptor-Dependent Immune Responses

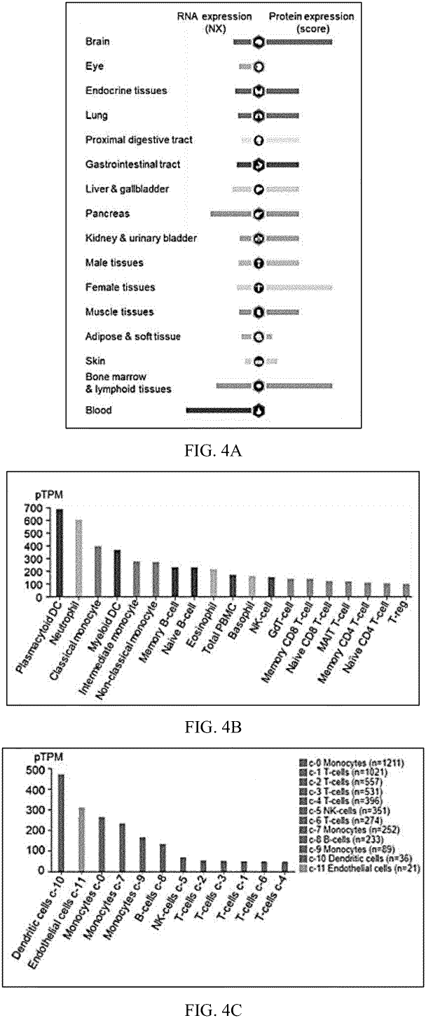

DENV’s affinity for cells and tissues affects DENV infection. To explore the role of CNPY3 in DENV infection, Human Platelet Antigen (HPA) database is used (https://www.proteinatlas.org/) to study the expression of CNPY3 in healthy human tissues. Data shows that RNA and protein of CNPY3 are more abundant in brain, blood, bone marrow and lymphoid tissues (FIG. 4A). The immune system, liver, and blood endothelial cells lining organ system play an important role in the pathogenesis of DHF/DSS. HPA database is used to further detect the expression of CNPY3 in various immune cells in blood.

Single cell sequencing data obtained from the HPA database shows that CNPY3 is most expressed in dendritic cells (DCs), endothelial cells, and monocytes in blood (FIGS. 4B and 4C). Monocytes and dendritic cells are the main target cells of DENV. The high expression of CNPY3 in these organs and cells shows that the CNPY3 plays an important role in the anti-dengue virus immune system.

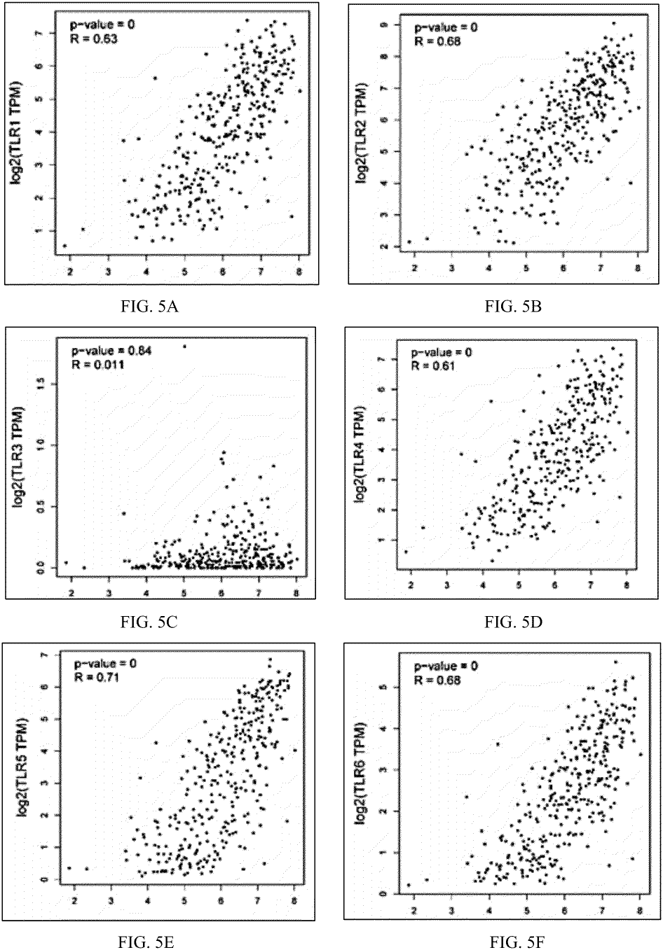

TLRs (Toll-like receptors) are key proteins for host defense, and participate in the host’s innate immune response to microbial invasion. CNPY3 is required for normal folding and expression of TLRs by gp96 (also referred to as glucose-regulated protein 94 abbreviated GRP94) in addition to Toll-like receptor 3 (TLR3). This suggests that CNPY3 may resist dengue virus infection through the innate immune response signaling pathways. The Gene Expression Profiling Interactive Analysis (GEPIA) database (http://gepia.cancel-pku.cn/) is used to explore the correlation between CNPY3 expression and TLRs expression. The GEPIA database shows that in the whole blood of healthy people, except for TLR3, the expressions of TLR1, TLR2, TLR4, TLR5, TLR6, TLR7, TLR8, TLR9, TLR10 and other TLRs are positively correlated with the expression of CNPY3 (FIGS. 5A-5J).

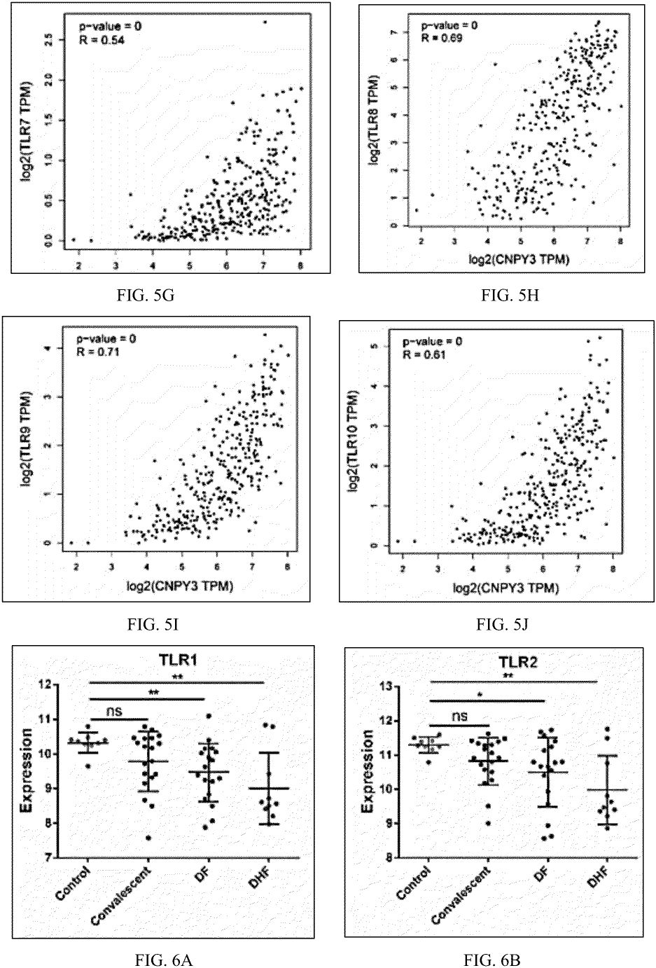

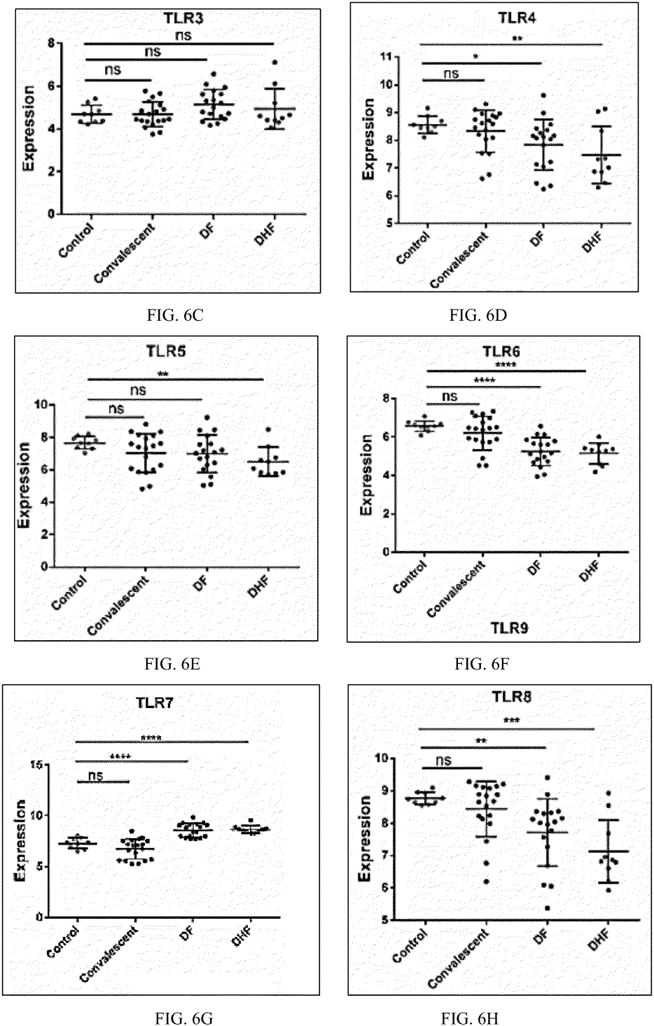

In addition, based on the dataset GSE51808, except for TLR3 and TLR7, the expressions of most TLRs are negatively correlated with the severity of dengue fever (FIGS. 6A-6J), which is consistent with the expression of CNPY3 in dengue patients.

TLRs signaling pathway promotes the productions of interferon type I, inflammatory factors and chemokines, and establishes antiviral immunity. In order to further explore the role of CNPY3 in antiviral immune response, small interfering RNA (siRNA) is used to inhibit the expression of CNPY3 in THP-1 cells, and after CNPY3 is down regulated, the content of antiviral interferon type I at downstream of the innate immune signaling pathway and the expression of antiviral related molecules are detected. The transient transfection of siRNA targeting CNPY3 into THP-1 cells is performed using Zeta Life Advanced DNA RNA transfection reagent (Zeta Life, USA) according to the instructions of the kit. The THP-1 cells are resuspended in a serum-containing complete medium and counted, and inoculated into a 6-well cell culture plate, 2 mL per well. The siRNA is synthesized by Sangon Biotech (Shanghai) Co., Ltd., with a sequence: sense with SEQ ID NO: 41 presented as 5′-gagcuguggaacgagacuucuutt-3′, and another sequence: antisense with SEQ ID NO: 42 presented as 5′-agaagucucucucucucucucucuut-3′. The volume of siRNA and transfection reagent is 1:1, and the synthesized negative control (NC) and siCNPY3 are dissolved in enzyme-free water to a final concentration of 20 µM. 8 µL NC/siCNPY3 and 8 µL of the transfection reagent of are gently mixed, incubated at room temperature for 15 minutes, and then added into the cell culture plate. After 36 hours of transfection, cells are collected for detection.

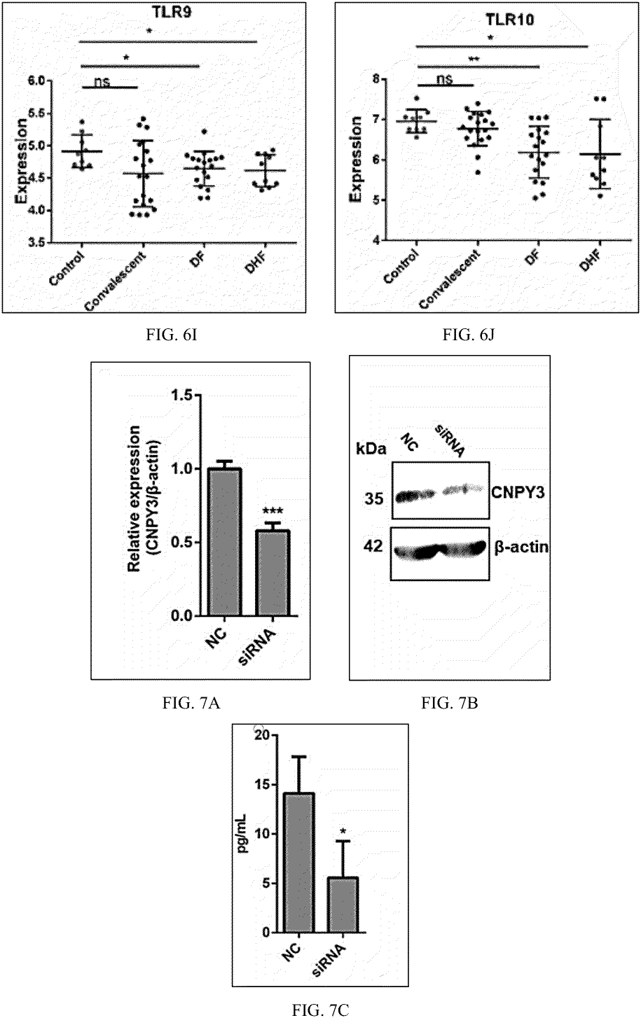

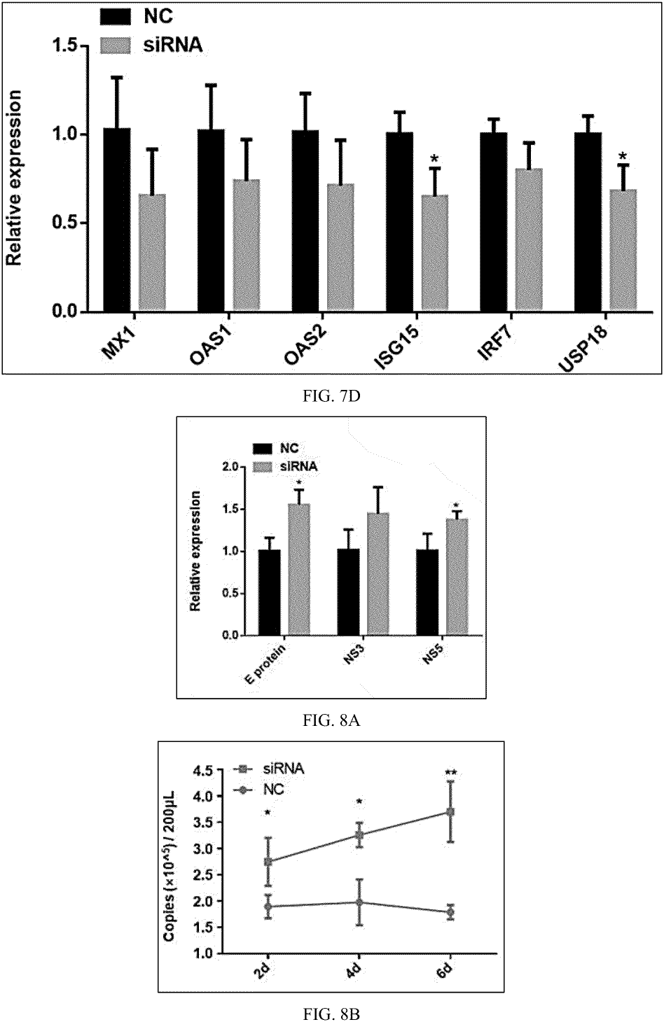

The results show that the use of siRNA targeting CNPY3 can significantly reduce the expression of mRNA and protein of CNPY3 in THP-1 cells (FIGS. 7A and 7B). The content of interferon type I IFN-β in supernatant of THP-1 cells transfected with siRNA targeting CNPY3 detected by using Huma IFN-β enzyme-linked immunosorbent assay (ELISA) kit (MultiSciences (Lianke) Biotech Co., Ltd., China). It is found that the down-regulation of CNPY3 results in inhibition of IFN-β production (FIG. 7C), and the related interferon-stimulated genes (ISGs) including MX1, OAS1, OAS2, ISG15, IRF7 and USP18 are down-regulated (FIG. 7D). Therefore, the down-regulation of CNPY3 will affect the antiviral immune response of the body.

### Embodiment 4 CNPY3 Inhibits the Replication of DENV-2

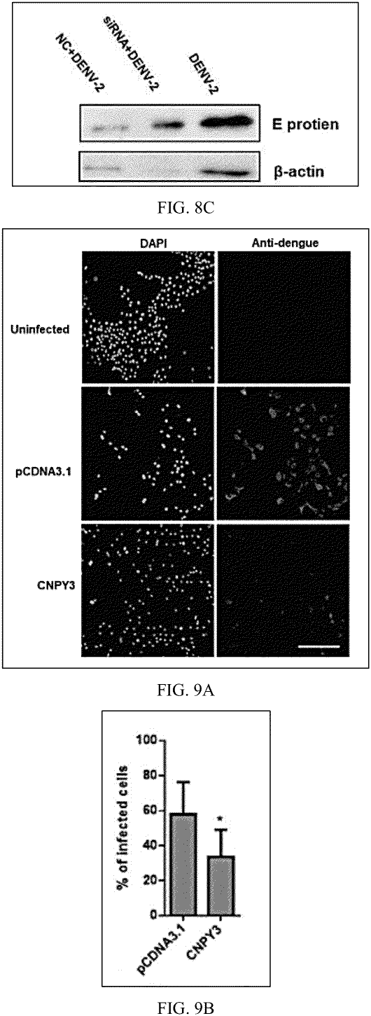

Considering the influence of CNPY3 knockdown on Toll-like receptor dependent-immune response, it is speculated that CNPY3 knockdown may increase DENV infection. To test this hypothesis, THP-1 cells are transfected with siRNA targeting CNPY3, and then infected with DENV-2 36 hours later. After 18 hours, the expressions of envelope proteins (also referred to as E proteins) and non-structural (NS) proteins NS3 and NS5 of DENV-2 in THP-1 cells are detected. As expected, compared with the control group, the down-regulation of CNPY3 protein enhances DENV-2 infection, the expressions of the genes of the envelope proteins and the non-structural proteins NS3 and NS5 of DENV-2 in THP-1 cells with down-regulation of CNPY3 are significantly higher than that in the control group (FIG. 8A). In addition, the virus content in the supernatant of cells infected with DENV-2 for 2, 4 and 6 days later shows that the number of virus copies of DENV-2 in THP-1 cells with down-regulation of CNPY3 is significantly higher than that in the control group (FIG. 8B). Western blot results show that the envelope proteins of DENV-2 are more in the supernatant of CNPY3-interfered THP-1 cells 6 days after DENV-2 infection (FIG. 8C).

The down-regulation of CNPY3 expression during DENV infection suggests that DENV may inhibit the downstream of the Toll-like anti-virus signaling pathway of the innate immune system by inhibiting the expression of CNPY3, thus escaping the surveillance of the innate immune system of the body to facilitate virus replication. It is speculated that increasing the expression of CNPY3 will inhibit the replication of DNEV. Therefore, an empty vector control (pcDNA3.1) or CNPY3 overexpression plasmid (pcDNA3.1-CNPY3) is constructed. Then, transient transfection is performed by using Lipofectamine™ 3000 transfection reagents (Invitrogen, USA), following the instructions of the kit. Cells are inoculated into a 6-well cell culture plate at 3×10⁵ /mL, 2 mL per well, cultured overnight in a 37° C.-incubator containing 5% carbon dioxide (CO₂), and transfection is performed when the cell density is 70%. 2 micrograms (µg) CNPY3/PCDNA3.1 plasmid is dulited with 125 µL serum-free high sugar dulbecco’s modified eagle medium (DMEM) (containing 5 µL P3000 Reagent), and 7.5 µL Lipofectamine™ 3000 transfection reagent is diluted with the same volume of serum-free high sugar DMEM. The diluted plasmid is added into the transfection reagent and gently mixed, and placed at room temperature for 15 minutes. The complete medium in the cell culture plate is replaced with the serum-free high sugar DMEM, and then the mixed solution is added to the cell culture plate. After 24 hours, the medium is replaced with fresh-complete culture medium, and the cells are collected for detection after 36 hours of transfection.

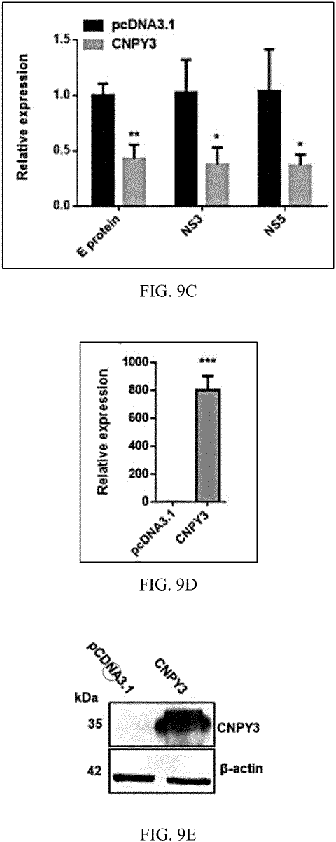

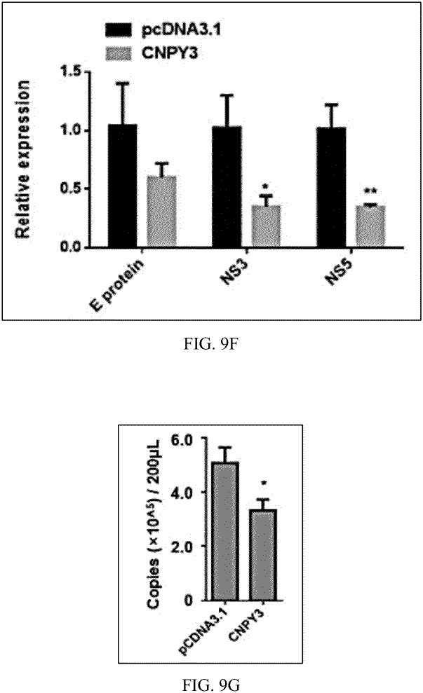

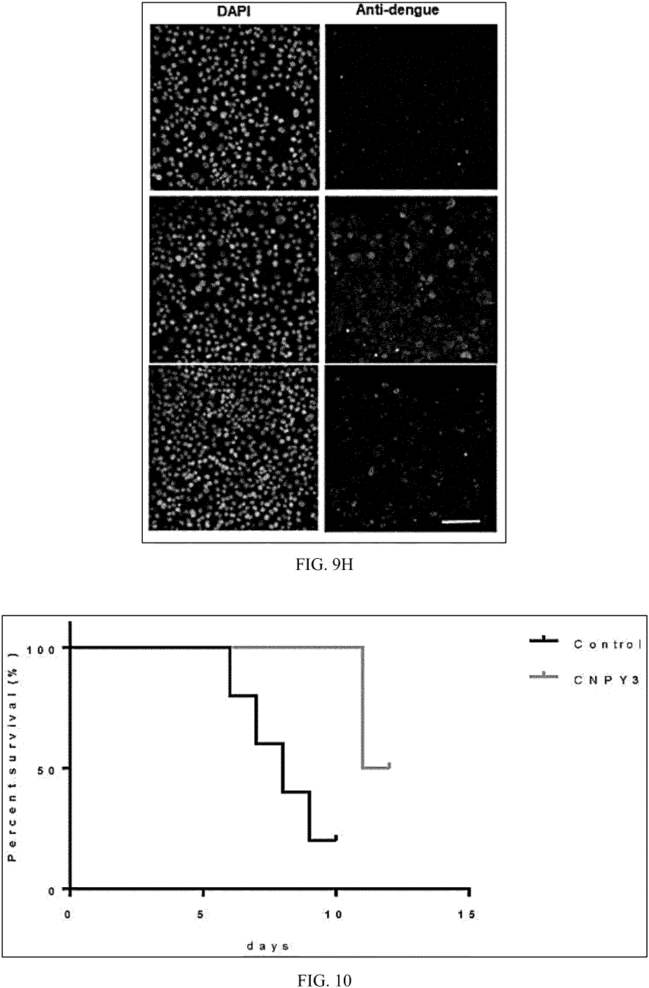

Vero cells are performed with transient transfection for 36 hours, then infected with DENV-2 (multiplicity of infection abbreviated MOI=2), and the virus content is detected by immunofluorescence 18 hours later. The specific implementation method is as follows. Vero cells are inoculated on a 24-well tissue culture plate and transfected with pcDNA3.1-CNPY3 or pcDNA3.1 empty vector plasmid when the cell density is 50%. Vero cells transfected for 36 hours are infected with DENV-2 (MOI=5) for 18 hours. Subsequently, the cells are fixed with 4% paraformaldehyde solution at room temperature for 15 minutes, and then treated with 0.5% Triton X-100 solution (preheated to 37° C.) for 10 minutes. The treated cells are rinsed twice with phosphate buffered saline (PBS), then sealed with 10% goat serum, incubated at room temperature for 30 minutes, and the sealing solution is sucked out with filter paper. 200 µL (1:100 dilution) of anti-Dengue virus 1+2+3+4 antibody (Abcam, England, ab26837) is added dropwise and placed into a wet box, and incubated overnight at 4° C. The incubated cells are rinsed twice with phosphate-buffered saline with Tween™ 20 (PBS-T), the water is absorbed with filter paper, and 200 µL (1:500 dilution) Alexa Fluor® 488 labeled goat anti-rabbit IgG (H+L) secondary antibody (Abcam, England, ab150077) is added dropwise and placed, incubated at 37° C. for 1 hour. After rinsing twice with PBS-T, the water is absorbed with filter paper, and 200 µL of 4′,6-diamidino-2-phenylindole (DAPI) (Beyotime Biotechnology, China) is added dropwise, the cells are incubated at room temperature for 5 minutes, rinsed twice with PBS, and then observed and photographed under a fluorescence microscope (Olympus IX53, Germany). Image J (NIH) software is used to count the number of DENV-infected cells in specific areas. The total percentage of Vero cells infected with DENV-2 is observed by a fluorescence microscope, and the expression of envelope proteins and non-structural proteins NS3 and NS5 of DENV-2 is detected by real-time fluorescent quantitative PCR. The results show that DENV-2 infection in Vero cells overexpressing CNPY3 protein is significantly inhibited compared with the empty vector control (FIGS. 9A, 9B and 9C). Since Vero cells are interferon-deficient cells, the anti-dengue virus effect of CNPY3 is further verified in HEK293T cells. Consistent with data of the Vero cell, the overexpression of CNPY3 protein in HEK 293T cells also results in a significant reduction in DENV-2 infection (FIGS. 9D, 9E, and 9F).

HEK 293T cells are performed with transient transfection with pCDNA3. 1-CNPY3 or pCDNA3.1 empty vector plasmid or THP-1 cells are performed with transient transfection with siRNA targeting CNPY3 for 36 hours, and then infected with DENV-2 (MOI=2). After DENV-2 infection for 2, 4 and 6 days, 200 µL of cell culture supernatant is collected, and viral RNA is extracted using a MiniBEST Viral RNA/DNA Extraction Kit (Takara, Japan). RNA is eluted with 30 µL of RNase-free water. Subsequently, 7 µL of RNA is taken for reverse transcription using Prime Script™ RT Reagent Kit (TaKaRa, Japan), and a total of 20 uL of cDNA is obtained. 1 uL cDNA is taken as template for quantitative PCR. Quantitative PCR primers are DV2-F: 5′-gcagaatgcccacacaaa-3′ (SEQ ID NO: 43) and DV2-R: 5′-acaaatacatccccctttct-3′ (SEQ ID NO: 44). The PCR amplification procedures are as follows: pre-denaturation at 95° C. for 3 minutes, denaturation at 95° C. for 10s; annealing at 57° C. for 30s; extension at 72° C. for 30s; and 42 cycles in total. PUC57 positive plasmid containing DENV-2NS 1 gene (synthesized by GenScript Biotech Corporation, Shanghai) is used for RT-PCR to establish a standard curve, and the copy number of DENV-2 is calculated with reference to the method of Lee et al. (i.e., Changsoo Lee, Jaai Kim, Seung Gu Shin, Seokhwan Hwang, “Absolute and relative QPCR quantification of plasmid copy number in Escherichia coli”, Journal of Biotechnology, 2006, pages 273-280, Volume 123, Issue 3). The number of virus copies per 200 uL of the sample is calculated from the quantitative PCR Cq value according to the standard curve. The results are shown in FIG. 9G, indicating that after CNPY3 is overexpressed in HEK 293T cells, the virus content is significantly reduced compared with the empty vector control.

After overexpression of CNPY3 protein in HEK 293T cells, the supernatant of cells infected with DENV-2 (MOI=2) for 6 days is collected and the virus content is detected by immunofluorescence analysis. As shown in FIG. 9H, the number of viruses in Vero cells transfected with pcDNA3.1-CNPY3 is significantly lower than that in Vero cells transfected with pcDNA3.1 empty vector plasmid, indicating that CNPY3 has obvious antiviral function.

### Embodiment 5 Animal Experiment

(1), Preparation of mRNA of CNPY3 is as follows. PUC57-mCNPY3 plasmid synthesized by GenScript Biotech Corporation, Shanghai.

SEQ ID NO: 45 represents a sequence of mCNPY3, and is specifically shown as follows:

gcagtcccggaagcggccgggggaagctgctccgcgcgcgctgccggagg
aagcgccgccgggtccgctctgctctgggtccggctgggccgccaccatg
gagtccatgtctgagctcgcgccccgctgcctcttatttcctttgctgct
gctgcttccgctgctgctccttcctgccccgaagctaggcccgagtcccg
ccggggctgaggagaccgactgggtgcgattgcccagcaaatgcgaagtg
tgcaagtatgttgctgtggagctgaagtcggcttttgaggaaacgggaaa
gaccaaggaagtgattgacaccggctatggcatcctggacgggaagggct
ctggagtcaagtacaccaagtcggacttacggttaattgaagtcactgag
accatttgcaagaggcttctggactacagcctgcacaaggagaggactgg
cagcaaccggtttgccaagggtatgtcggagacctttgagacgctgcaca
acctagtccacaaaggggtcaaggtggtgatggatatcccctatgagctg
tggaacgagacctcagcagaggtggctgacctcaagaagcagtgtgacgt
gctggtggaagagtttgaagaggtgattgaggactggtacaggaaccacc
aggaggaagacctgactgaattcctctgtgccaaccacgtgctgaaggga
aaggacacgagttgcctagcagagcggtggtctggcaagaagggggacat
agcctccctgggagggaagaaatccaagaagaagcgcagcggagtcaagg
gctcctccagtggcagcagcaagcagaggaaggaactggggggcctgggg
gaggatgccaacgccgaggaggaggagggtgtgcagaaggcatcgcccct
cccacacagcccccctgatgagctgtgattgtgtatgcgttaataaaaag
aaggaactcgta

Plasmids are linearized using Sal enzyme (TakaRa, Japan), in vitro transcription is performed by using the In Vitro Transcription Kit (T7 High Yield RNA Transcription kit, Cat. No.: E131-01A from Suzhou Novoprotein Technology Co., Ltd.), and a capping kit (mRNA Cap 2′-O-Methyltransferase, article No.: M072) and a tailing kit (E.coli Poly(A) Polymerase, article No.: M012) are used for adding a cap structure and A-tailing. Then, lipid nanoparticles (LNPs) are used for encapsulation.

(2), during intracranial challenge, 10 µL of DENV-2 (60 PFU) is taken and mixed with 10 µL of empty vector LNP and 1 µg of mCNPY3 LNP individually, and then injected simultaneously into 5 or 6 3-day-old suckling mice. The survival rate is then calculated daily. The results show that CNPY3 has a protective effect on DENV-2 infected suckling mice (FIG. 10).

All the above results indicate that promotes DENV invasion and spreading by inhibiting the expression of CNPY3, which in turn disrupts Toll-like receptor-dependent immune responses. CNPY3 has an inhibitory effect on DENV replication. The mCNPY3 is made into an mRNA preparation in a mouse body to carry out intracranial challenge on suckling mice together with the virus, which has a protective effect on the suckling mice.

The disclosure uses the disclosed dataset to analyze genes related to dengue virus infection and identify an important host protein CNPY3. The host gene is significantly down-regulated in the blood and in dengue virus infected dendritic cells and THP-1 cells of dengue patients. The expression of CNPY3 is negatively correlated with the progression of dengue disease and positively correlated with the expression of most Toll-like receptors. Further studies show that down-regulation of the host gene can inhibit the production of IFN-β and the expression of interferon-stimulated genes (ISGs), and promote DENV-2 infection. However, up-regulation of the host gene expression in Vero and HEK 293T cells can inhibit DENV-2 infection. These results indicate that CNPY3 participates in the innate immune response signaling pathway, has the effect of anti-dengue virus, and is a potential therapeutic target for dengue fever.

What is claimed is:

1. A use of a canopy fibroblast growth factor signaling regulator 3 (CNPY3) protein, comprising:
taking the CNPY3 protein as a target to treat dengue fever.

2. The use according to claim 1, wherein an amino acid sequence of the CNPY3 protein is as shown in SEQ ID NO: 1.

3. The use according to claim 2, wherein a nucleotide sequence of an encoding gene of the CNPY3 protein is as shown in one of SEQ ID NO: 2 and SEQ ID NO: 45.

4. A use of a CNPY3 protein, comprising:
upregulating an expression of the CNPY3 protein in an anti-dengue virus.

5. The use according to claim 4, wherein an amino acid sequence of the CNPY3 protein is as shown in SEQ ID NO: 1.

6. The use as claimed in claim 5, wherein a nucleotide sequence of an encoding gene of the CNPY3 protein is as shown in one of SEQ ID NO: 2 and SEQ ID NO: 45.

7. An anti-dengue virus drug, comprising:
at least one of a messenger ribonucleic acid (mRNA) of CNPY3, a CNPY3 protein, a carrier expressing CNPY3 protein, a host cell expressing CNPY3 protein.

8. The drug according to claim 7, wherein an amino acid sequence of the CNPY3 protein is as shown in SEQ ID NO: 1.

9. The drug according to claim 8, wherein a nucleotide sequence of an encoding gene of the CNPY3 protein is as shown in one of SEQ ID NO: 2 and SEQ ID NO: 45.

10. The drug according to claim 9, wherein the mRNA of CNPY3 is encapsulated with lipid nanoparticles (LNPs).

11. The drug according to claim 10, wherein the mRNA of CNPY3 protein is cloned onto a T/A carrier, transcribed in vitro after linearization, the transcribed mRNA is capped and tailed, and encapsulated with the LNPs.


SEQUENCE LISTING (45 sequences)

SEQ ID NO: 1 - skipped

SEQ ID NO: 2 - DNA, 837 bp, synthetic construct
  source 1..837: mol_type = other DNA
atggattcaatgcctgagcccgcgtcccgctgtcttctgcttcttcccttgctgctgctgctgctgctgctgctgccggccccggagctgggcccgagccaggccggagctgaggagaacgactgggttcgcctgcccagcaaatgcgaagtgtgtaaatatgttgctgtggagctgaagtcagcctttgaggaaaccggcaagaccaaggaggtgattggcacgggctatggcatcctggaccagaaggcctctggagtcaaatacaccaagtcggacttgcggttaatcgaagtcactgagaccatttgcaagaggctcctggattatagcctgcacaaggagaggaccggcagcaatcgatttgccaagggcatgtcagagacctttgagacattacacaacctggtacacaaaggggtcaaggtggtgatggacatcccctatgagctgtggaacgagacttctgcagaggtggctgacctcaagaagcagtgtgatgtgctggtggaagagtttgaggaggtgatcgaggactggtacaggaaccaccaggaggaagacctgactgaattcctctgcgccaaccacgtgctgaagggaaaagacaccagttgcctggcagagcagtggtccggcaagaagggagacacagctgccctgggagggaagaagtccaagaagaagagcagcagggccaaggcagcaggcggcaggagtagcagcagcaaacaaaggaaggagctgggtggccttgagggagaccccagccccgaggaggatgagggcatccagaaggcatcccctctcacacacagcccccctgatgagctctga

SEQ ID NO: 3 - DNA, 22 bp, synthetic construct
  source 1..22: mol_type = other DNA
caccattggcaatgagcggttc

SEQ ID NO: 4 - DNA, 22 bp, synthetic construct
  source 1..22: mol_type = other DNA
aggtctttgcggatgtccacgt

SEQ ID NO: 5 - DNA, 22 bp, synthetic construct
  source 1..22: mol_type = other DNA
caccattggcaatgagcggttc

SEQ ID NO: 6 - DNA, 22 bp, synthetic construct
  source 1..22: mol_type = other DNA
aggtctttacggatgtccacgt

SEQ ID NO: 7 - DNA, 21 bp, synthetic construct
  source 1..21: mol_type = other DNA
tgctctcacctcatcagcagt

SEQ ID NO: 8 - DNA, 22 bp, synthetic construct
  source 1..22: mol_type = other DNA
cacaactcctccaatcacaact

SEQ ID NO: 9 - DNA, 21 bp, synthetic construct
  source 1..21: mol_type = other DNA
gtttccgaagtggacatcgca

SEQ ID NO: 10 - DNA, 20 bp, synthetic construct
  source 1..20: mol_type = other DNA
ctgcacaggttgttctcagc

SEQ ID NO: 11 - DNA, 21 bp, synthetic construct
  source 1..21: mol_type = other DNA
tgtccaaggtggtaaagggtg

SEQ ID NO: 12 - DNA, 21 bp, synthetic construct
  source 1..21: mol_type = other DNA
ccggcgatttaactgatcctg

SEQ ID NO: 13 - DNA, 22 bp, synthetic construct
  source 1..22: mol_type = other DNA
ccttacagcgaacctcatccac

SEQ ID NO: 14 - DNA, 22 bp, synthetic construct
  source 1..22: mol_type = other DNA
tccactacaggaggagaagcga

SEQ ID NO: 15 - DNA, 22 bp, synthetic construct
  source 1..22: mol_type = other DNA
gtcaaggtggtgatggacatcc

SEQ ID NO: 16 - DNA, 22 bp, synthetic construct
  source 1..22: mol_type = other DNA
ctgtaccagtcctcgatcacct

SEQ ID NO: 17 - DNA, 24 bp, synthetic construct
  source 1..24: mol_type = other DNA
gtggagaatacaaatgtcagcacc

SEQ ID NO: 18 - DNA, 22 bp, synthetic construct
  source 1..22: mol_type = other DNA
ctccatcaccacctcagcagag

SEQ ID NO: 19 - DNA, 23 bp, synthetic construct
  source 1..23: mol_type = other DNA
gcttccgacaatcaacagccaag

SEQ ID NO: 20 - DNA, 22 bp, synthetic construct
  source 1..22: mol_type = other DNA
cttgacgattttgtgccgctcg

SEQ ID NO: 21 - DNA, 22 bp, synthetic construct
  source 1..22: mol_type = other DNA
ctctgagcatcctggtgaggaa

SEQ ID NO: 22 - DNA, 22 bp, synthetic construct
  source 1..22: mol_type = other DNA
aaggtcagccagaacaggtcgt

SEQ ID NO: 23 - DNA, 22 bp, synthetic construct
  source 1..22: mol_type = other DNA
ccacgctataccatctacctgg

SEQ ID NO: 24 - DNA, 22 bp, synthetic construct
  source 1..22: mol_type = other DNA
gctgctatccagggaagacaca

SEQ ID NO: 25 - DNA, 22 bp, synthetic construct
  source 1..22: mol_type = other DNA
tggacagacctgctgccttaac

SEQ ID NO: 26 - DNA, 22 bp, synthetic construct
  source 1..22: mol_type = other DNA
ctgtcctgcatcttctccagca

SEQ ID NO: 27 - DNA, 20 bp, synthetic construct
  source 1..20: mol_type = other DNA
ttcggtctgcattcccagtg

SEQ ID NO: 28 - DNA, 22 bp, synthetic construct
  source 1..22: mol_type = other DNA
aacttaaattggggcaagtggc

SEQ ID NO: 29 - DNA, 20 bp, synthetic construct
  source 1..20: mol_type = other DNA
tggagctgaagtcggctttt

SEQ ID NO: 30 - DNA, 20 bp, synthetic construct
  source 1..20: mol_type = other DNA
cttgtgcaggctgtagtcca

SEQ ID NO: 31 - DNA, 20 bp, synthetic construct
  source 1..20: mol_type = other DNA
aggagtagagccgggacaat

SEQ ID NO: 32 - DNA, 20 bp, synthetic construct
  source 1..20: mol_type = other DNA
cgctcccctcattgttgtct

SEQ ID NO: 33 - DNA, 20 bp, synthetic construct
  source 1..20: mol_type = other DNA
agcccatttcacagacccag

SEQ ID NO: 34 - DNA, 20 bp, synthetic construct
  source 1..20: mol_type = other DNA
tgtccagaactccacgaacg

SEQ ID NO: 35 - DNA, 20 bp, synthetic construct
  source 1..20: mol_type = other DNA
gatgtagacctcggaagcgg

SEQ ID NO: 36 - DNA, 20 bp, synthetic construct
  source 1..20: mol_type = other DNA
gtcagcagtctgaccactcc

SEQ ID NO: 37 - DNA, 20 bp, synthetic construct
  source 1..20: mol_type = other DNA
gcagaatgccccaacacaaa

SEQ ID NO: 38 - DNA, 24 bp, synthetic construct
  source 1..24: mol_type = other DNA
acaaaatacatcctgcctttctct

SEQ ID NO: 39 - DNA, 20 bp, synthetic construct
  source 1..20: mol_type = other DNA
gcagaatgccccaacacaaa

SEQ ID NO: 40 - DNA, 24 bp, synthetic construct
  source 1..24: mol_type = other DNA
acaaaatacatcctgcctttctct

SEQ ID NO: 41-42 - skipped (2 entries)

SEQ ID NO: 43 - DNA, 20 bp, synthetic construct
  source 1..20: mol_type = other DNA
gcagaatgccccaacacaaa

SEQ ID NO: 44 - DNA, 24 bp, synthetic construct
  source 1..24: mol_type = other DNA
acaaaatacatcctgcctttctct

SEQ ID NO: 45 - DNA, 962 bp, synthetic construct
  source 1..962: mol_type = other DNA
gcagtcccggaagcggccgggggaagctgctccgcgcgcgctgccggaggaagcgccgccgggtccgctctgctctgggtccggctgggccgccaccatggagtccatgtctgagctcgcgccccgctgcctcttatttcctttgctgctgctgcttccgctgctgctccttcctgccccgaagctaggcccgagtcccgccggggctgaggagaccgactgggtgcgattgcccagcaaatgcgaagtgtgcaagtatgttgctgtggagctgaagtcggcttttgaggaaacgggaaagaccaaggaagtgattgacaccggctatggcatcctggacgggaagggctctggagtcaagtacaccaagtcggacttacggttaattgaagtcactgagaccatttgcaagaggcttctggactacagcctgcacaaggagaggactggcagcaaccggtttgccaagggtatgtcggagacctttgagacgctgcacaacctagtccacaaaggggtcaaggtggtgatggatatcccctatgagctgtggaacgagacctcagcagaggtggctgacctcaagaagcagtgtgacgtgctggtggaagagtttgaagaggtgattgaggactggtacaggaaccaccaggaggaagacctgactgaattcctctgtgccaaccacgtgctgaagggaaaggacacgagttgcctagcagagcggtggtctggcaagaagggggacatagcctccctgggagggaagaaatccaagaagaagcgcagcggagtcaagggctcctccagtggcagcagcaagcagaggaaggaactggggggcctgggggaggatgccaacgccgaggaggaggagggtgtgcagaaggcatcgcccctcccacacagcccccctgatgagctgtgattgtgtatgcgttaataaaaagaaggaactcgta